(19) United States
(12) Patent Application Publication
(10) Pub. No.: US 2014/0287231 A1
(43) Pub. Date: Sep. 25, 2014

(54) Comfy Tape

(71) Applicant: Brian M. Paul, (US)

(72) Inventor: Brian M. Paul, Dallas, TX (US)

(73) Assignee: Brian M. Paul, Dallas, TX (US)

(21) Appl. No.: 13/848,629

(22) Filed: Mar. 21, 2013

(51) Int. Cl. A61L 24/00

(52) U.S. Cl. CPC A61L 24/00
USPC 428/343

(57) ABSTRACT

A tape device to be applied to a user may include a first tape portion to be applied to a skin of the user and a second tape portion to be connected to the first tape portion. The first tape portion may be covered by a first film of first adhesive and the second tape portion may be covered by a second film of second adhesive.

### FIELD OF THE INVENTION

The present invention relates to a tape and more particularly to a tape device to relieve discomfort.

### BACKGROUND

Most people at one time or another may have a pair of uncomfortable shoes which may cause discomfort to the top of the toes, the side of the foot or the bottom of the foot. This can cause discomfort for an entire day. There is a need for way of providing comfort from the discomfort of shoes and other sources of discomfort.

### SUMMARY

A tape device to be applied to a user may include a first tape portion to be applied to a skin of the user and a second tape portion to be connected to the first tape portion.

The first tape portion may be covered by a first film of first adhesive and the second tape portion may be covered by a second film of second adhesive.

The first adhesive may be pressure sensitive adhesive.

The first adhesive may be an hypo-allergenic pressure sensitive adhesive.

The first tape portion may be formed from silicone.

The first tape portion may be translucent.

The first tape portion may have a hardness softer than 10 durometer.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates a perspective view of the tape device of the present invention;

FIG. 2 illustrates a side view of the tape device of the present invention;

FIG. 3 illustrates a side view of the tape device of the present invention;

FIG. 4 illustrates a bottom view of the tape device of the present invention;

FIG. 5 illustrates a top view of the tape device of the present invention;

FIG. 6 illustrates an exploded view of the tape device of the present invention;

FIG. 7 illustrates a perspective view of the tape device of the present invention being used;

FIG. 8 illustrates a side view of another tape device of the present invention;

FIG. 9 illustrates a side view of the tape device of the present invention;

FIG. 10 illustrates a top view of a first layer of the tape device of the present invention;

FIG. 11 illustrates a top view of a fourth layer of the tape device of the present invention;

FIG. 12 illustrates a top view of the third layer of the tape device of the present invention;

FIG. 13 illustrates a top view of the second layer of the tape device of the present invention;

### DETAILED DESCRIPTION

The tape device 100 of the present invention may be an ultra-soft (within the range of hardness softer than 10 durometer), and the tape device 100 may be formed from a transparent, translucent or opaque silicone strip. The tape device 100 may include a first tape portion 103 (within the range of hardness softer than 10 durometer) which may be adapted to adhere to the skin of the user by a first film 203 of removable first adhesive which may be a hypo-allergenic pressure sensitive adhesive (PSA) (or other type of adhesive) which may be formed on the first bottom surface 107 of the first tape portion 103 and the second top surface 133 of the second tape portion 102 may include a second film 201 of removable second adhesive. The first bottom surface 107 may be directly connected to the skin surface of the user to remove discomfort from pressure, rubbing, binding etc.

The first tape portion 103 may be firmer (harder) than the second tape portion 102 to provide a firmer contact surface and to maintain the structure of the tape portion 100 as the tape portion 100 is being inserted into a shoe or other item. The tape device 100 may not be applied to open wound or infected areas. The tape device 100 may be placed on a shoulder of the user to relief where straps may cut into the skin or on the waist where the pants, shorts or belts may cause discomfort. The tape device 100 may relieve pressure, pain, tenderness and may help to prevent rubbing and blisters and provide relief for general discomfort.

The tape device 100 may be used to relieve pressure and rubbing from shoes caused by standing, walking and/or running.

The users of the tape device 100 may cut the tape device to a desired shape such as a rectangle, oval, circle or other appropriate shapes.

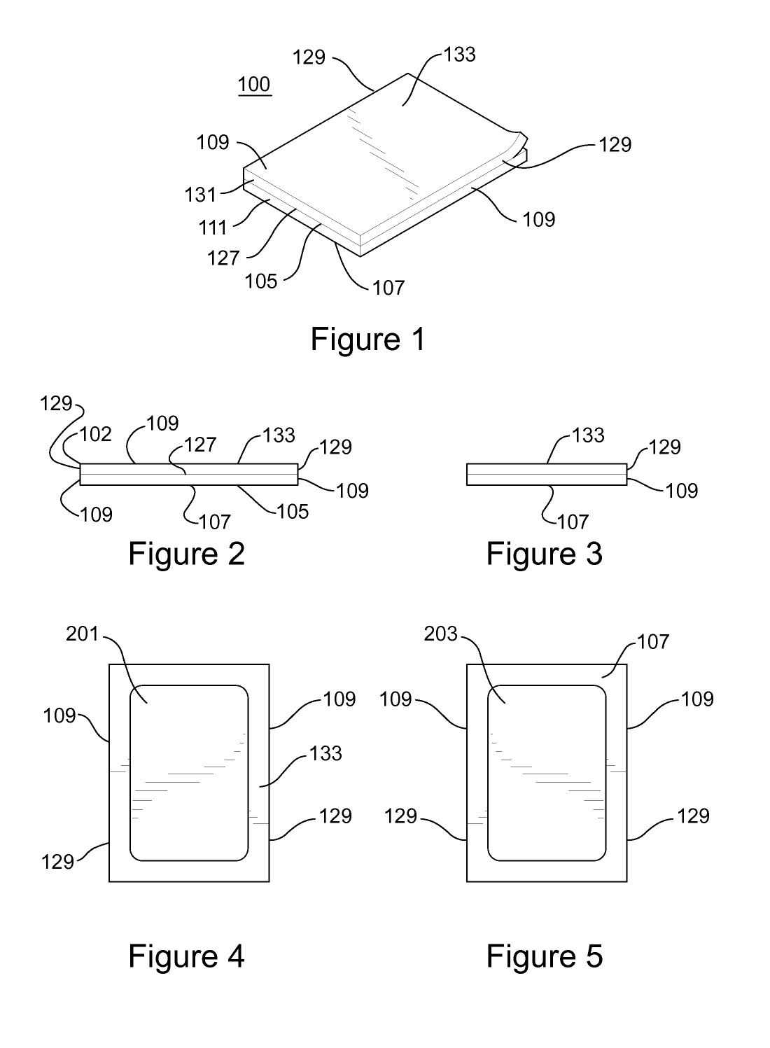

FIG. 1 illustrates a perspective view of the tape device 100 which may include a first tape device portion 103 and a second tape device portion 102. The first tape device portion 103 may include a front first surface 111 which may be opposed to a back first surface 113 and may be connected to a pair of opposing side first surfaces 109, a top first surface 105 and a bottom first surface 107, the top first surface 105 may be connected to the pair of opposing side first surfaces 109, the front first surface 111, and the back first surface 113, the bottom first surface 107 may be connected to the pair of opposing side first surfaces 109, the front first surface 111 and the back first surface 113, the back front surface 113 may be connected to the top first surface 105, the bottom first surface 107, and the side first surface 109, and the pair of side first surfaces 109 may be connected to the top first surface 105, the bottom first surface 107, front first surface 111 and the back first surface 103.

The second tape device portion 102 may include a front second surface 131 which may be opposed to a back second surface 133 and may be connected to a pair of opposing side second surfaces 129, a top second surface 125 and a bottom second surface 127, the top second surface 125 may be connected to the pair of opposing side second surfaces 129, the front second surface 131, and the back second surface 133, the bottom second surface 127 may be connected to the pair of opposing side second surfaces 129, the front second surface 131 and the back second surface 133, the back second surface 133 may be connected to the top second surface 125, the bottom second surface 127, and the side second surface 129, and the pair of side second surfaces 129 may be connected to the top second surface 125, the bottom second surface 127, front second surface 131 and the back second surface 133.

FIG. 2 illustrates a front view of the tape device 100 which may include a first tape device portion 103 and a second tape device portion 102. The first tape device portion 103 may include a front first surface 111 which may be opposed to a back first surface 113 and may be connected to a pair of opposing side first surfaces 109, a top first surface 105 and a bottom first surface 107, the top first surface 105 may be connected to the pair of opposing side first surfaces 109, the front first surface 111, and the back first surface 113, the bottom first surface 107 may be connected to the pair of opposing side first surfaces 109, the front first surface 111 and the back first surface 113, the back front surface 113 may be connected to the top first surface 105, the bottom first surface 107, and the side first surface 109, and the pair of side first surfaces 109 may be connected to the top first surface 105, the bottom first surface 107, front first surface 111 and the back first surface 103.

The second tape device portion 102 may include a front second surface 131 which may be opposed to a back second surface 133 and may be connected to a pair of opposing side second surfaces 129, a top second surface 125 and a bottom second surface 127, the top second surface 125 may be connected to the pair of opposing side second surfaces 129, the front second surface 131, and the back second surface 133, the bottom second surface 127 may be connected to the pair of opposing side second surfaces 129, the front second surface 131 and the back second surface 133, the back second surface 133 may be connected to the top second surface 125, the bottom second surface 127, and the side second surface 129, and the pair of side second surfaces 129 may be connected to the top second surface 125, the bottom second surface 127, front second surface 131 and the back second surface 133.

The first top surface 105 of the first tape portion 103 may be connected to the second bottom surface 127.

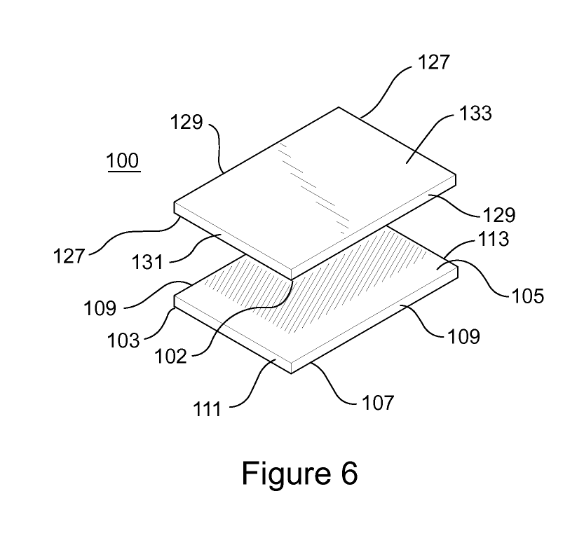

FIG. 6 illustrates an exploded view of the tape device 100 which may include a first tape device portion 103 and a second tape device portion 102. The first tape device portion 103 may include a front first surface 111 which may be opposed to a back first surface 113 and may be connected to a pair of opposing side first surfaces 109, a top first surface 105 and a bottom first surface 107, the top first surface 105 may be connected to the pair of opposing side first surfaces 109, the front first surface 111, and the back first surface 113, the bottom first surface 107 may be connected to the pair of opposing side first surfaces 109, the front first surface 111 and the back first surface 113, the back front surface 113 may be connected to the top first surface 105, the bottom first surface 107, and the side first surface 109, and the pair of side first surfaces 109 may be connected to the top first surface 105, the bottom first surface 107, front first surface 111 and the back first surface 103.

The second tape device portion 102 may include a front second surface 131 which may be opposed to a back second surface 133 and may be connected to a pair of opposing side second surfaces 129, a top second surface 125 and a bottom second surface 127, the top second surface 125 may be connected to the pair of opposing side second surfaces 129, the front second surface 131, and the back second surface 133, the bottom second surface 127 may be connected to the pair of opposing side second surfaces 129, the front second surface 131 and the back second surface 133, the back second surface 133 may be connected to the top second surface 125, the bottom second surface 127, and the side second surface 129, and the pair of side second surfaces 129 may be connected to the top second surface 125, the bottom second surface 127, front second surface 131 and the back second surface 133.

FIG. 3 illustrates a side view of the side first surface 109, the side second surface 129, the top second surface 133 and the first bottom surface 107.

FIG. 4 illustrates a top view of the tape device 100 and illustrates the top second surface 103, the side first surface 109 and the side second surface 129. FIG. 4 additionally illustrates the second film 201.

FIG. 5 illustrates a bottom view of the tape device 100 and illustrates the bottom first surface 107, the side first surface 109 and the second side surface 129. FIG. 5 additionally illustrates the first film 203.

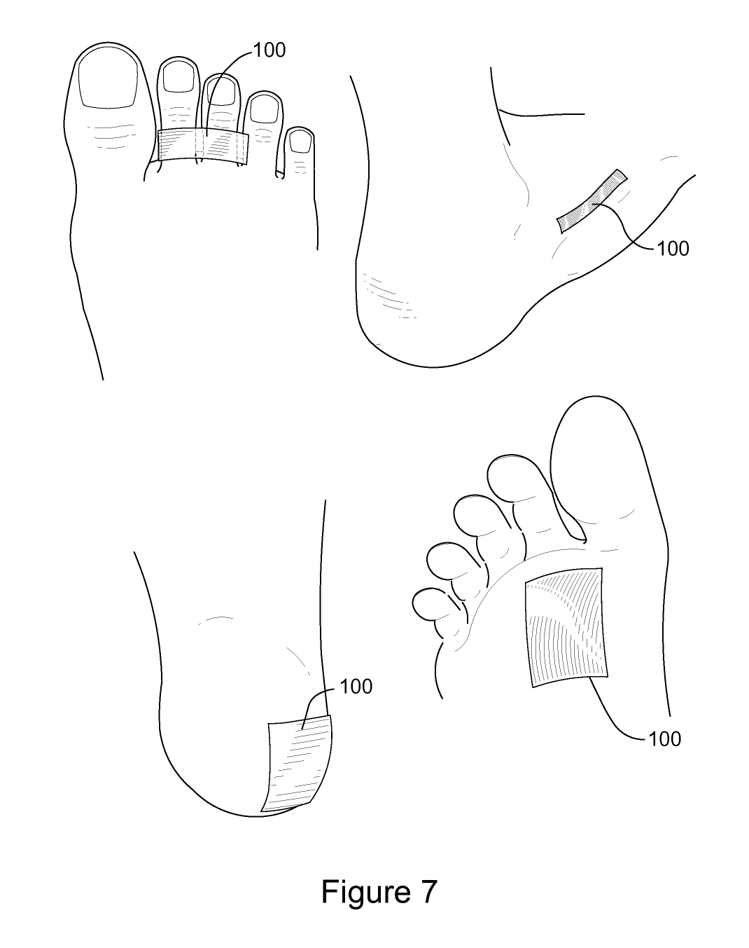

FIG. 7 illustrates the tape device 100 being used on various parts of the user.

The tape device 100 of the present invention may be an ultra-soft (within the range of hardness softer than 10 durometer), and the tape device 100 may be formed from a transparent, translucent or opaque silicone strip. The tape device 100 may include a first tape portion 103 which may be adapted to adhere to the skin of the user by a first film 203 of removable adhesive which may be a hypo-allergenic pressure sensitive adhesive (PSA) (or other type of adhesive) which may be formed on the first bottom surface 107 of the first tape portion 103 and the second top surface 133 of the second tape portion 102 may include a second film 201 of removable adhesive. The first bottom surface 107 may be directly connected to the skin surface of the user to remove discomfort from pressure, rubbing, binding etc.

The first tape portion 103 may be firmer (harder) than the second tape portion 102 to provide a firmer contact surface and to maintain the structure of the tape portion 100 as the tape portion 100 is being inserted into a shoe or other item. The tape device 100 may not be applied to open wound or infected areas. The tape device 100 may be placed on a shoulder of the user to relief where straps may cut into the skin or on the waist where the pants, shorts or belts may cause discomfort. The tape device 100 may relieve pressure, pain, tenderness and may help to prevent rubbing and blisters and provide relief for general discomfort.

The tape device 100 may be used to relieve pressure and rubbing from shoes caused by standing, walking and/or running.

The users of the tape device 100 may cut the tape device to a desired shape such as a rectangle, oval, circle or other appropriate shapes.

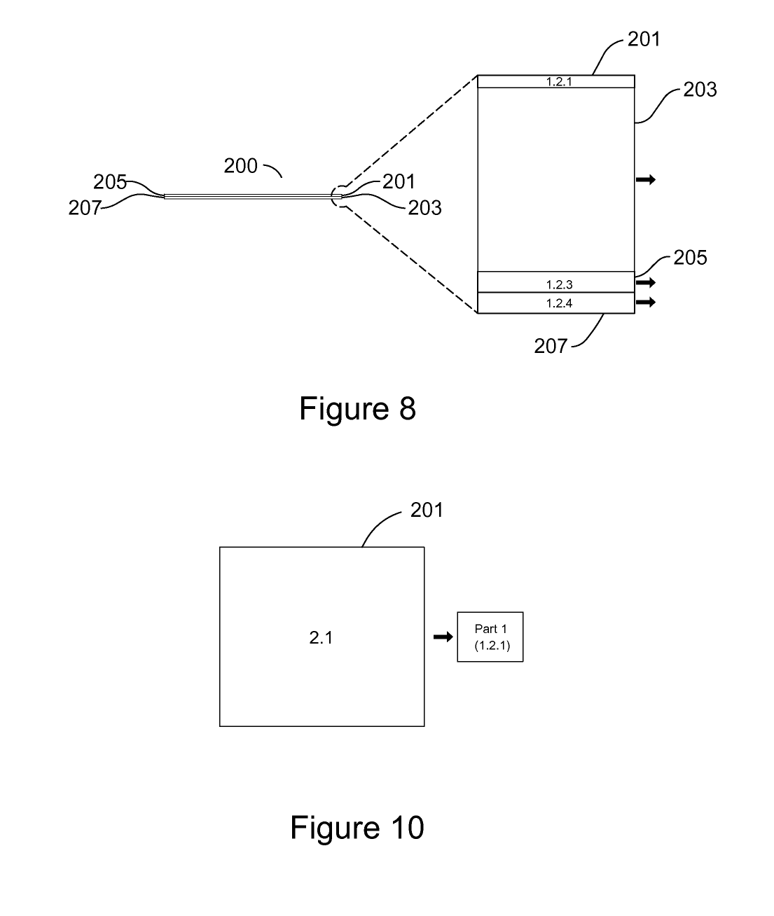

FIG. 8 illustrates a side view of the tape device 200 which may include a first layer 201 which may have a exterior surface, a second layer 203 which may be adjacent to the first layer 201, a third layer 205 which may be adjacent to the second layer 203 and a fourth layer 207 which may include a exterior surface and may be adjacent to the third layer 205.

FIG. 9 illustrates a side view of the tape device 200 which may include a first layer 201 which may have an exterior surface, a second layer 203 which may be adjacent to the first layer 201, a third layer 205 which may be adjacent to the second layer 203 and a fourth layer 207 which may include a exterior surface and may be adjacent to the third layer 205.

The first layer 201 as illustrated in FIG. 10 may be a top layer which may be formed from silicon, plastic or other appropriate material to isolate the second layer 203 from unintended attachments to skin or clothing.

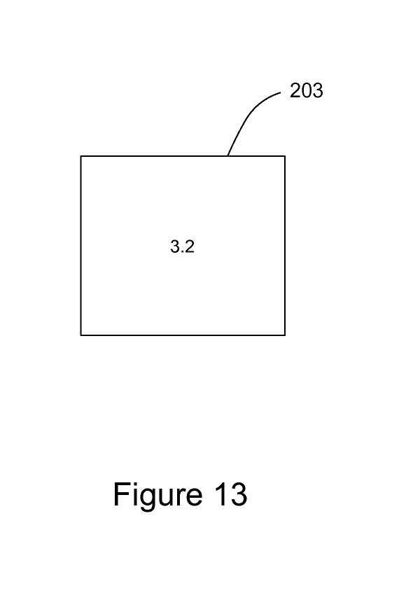

The second layer 203 as illustrated in FIG. 13 may be sandwiched between the first layer 201 and the third layer 205 and may be formed from a medical grade soft scar removal type of silicon gel which may provide anti-friction/rubbing relief and may provide a padding barrier to the user.

The third layer 205 as illustrated in FIG. 12 may be sandwiched between the second layer 203 and the fourth layer 207 and may be an adhesive layer of medical grade firm stick silicone adhesive gel which may be a removable adhesive to allow removal of the fourth layer 207 and to allow the third layer 205 to be detachably connected to the skin of the user.

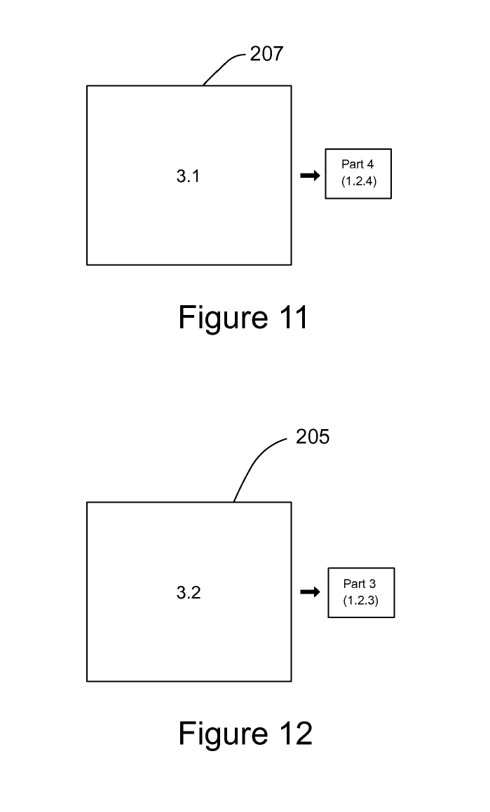

The fourth layer 207 as illustrated in FIG. 11 may be adjacent to the third layer 205 and may have an exterior surface and may include adhesive layer of medical grade firm stick silicone adhesive to detachably connect to the third layer 205.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1. A tape device to be applied to a user, comprising:
a first tape portion to be applied to a skin of the user;
a second tape portion to be connected to the first tape portion;
wherein the first tape portion is covered by a first film of first adhesive and wherein the second tape portion is covered by a second film of second adhesive.

2. A tape device to be applied to a user as in claim 1, wherein the first adhesive is pressure sensitive adhesive.

3. A tape device to be applied to a user as in claim 1, wherein the first adhesive is hypo-allergenic pressure sensitive adhesive.

4. A tape device to be applied to a user as in claim 1, wherein the first tape portion is formed from silicone.

5. A tape device to be applied to a user as in claim 1, wherein the first tape portion is translucent.

6. A tape device to be applied to a user as in claim 1, wherein the first tape portion has a hardness softer than 10 durometer.
